(19) United States
(12) Patent Application Publication
(10) Pub. No.: US 2002/0155164 A1
(43) Pub. Date: Oct. 24, 2002

(54) System and method for the prevention of infections in human patients using nitric oxide

(76) Inventors: Curtis Figley, Edmonton (CA); Doug R. Hole, Edmonton (CA); Christopher C. Miller, North Vancouver (CA); Alex Stenzler, Orange, CA (US)

Correspondence Address: Terry M. Gernstein, 1015 Salt Meadow Lane, Mclean, VA, 22101, US

(21) Appl. No.: 09/836,603

(22) Filed: Apr. 18, 2001

(51) Int. Cl. A61K 33/00; A61M 37/00

(52) U.S. Cl. 424/600; 604/23

(57) ABSTRACT

A system and method for topically treating a wound or lesion of tissue to prevent infection includes applying nitric oxide gas to the wound or lesion. The basic system includes a source of nitric oxide gas and a flushing envelope. The flushing envelope is applied to a patient to cover a wound or lesion and receives nitric oxide from the source via a flow control valve. One form of the system also includes a vacuum unit fluidically connected to the flushing envelope, and one embodiment includes a gas absorber unit. The flushing envelope is adapted to surround the area of the infected tissue and form a substantially airtight seal with the tissue surface when the flushing envelope is in place on the patient. The flow control valve controls the amount of nitric oxide gas that is delivered to the flushing envelope. A source of dilutant gas is fluidically connected to the flow control valve and a system control unit transmits and receives signals from various sensors and controlled elements in the system. NO and NO₂ sensors are included in one form of the system.

### TECHNICAL FIELD OF THE INVENTION

The present invention relates to the general art of surgery, and to the particular field of applying material to wounds for therapeutic purposes.

### BACKGROUND OF THE INVENTION

Wounds and lesions are encountered where infectious contaminants are. Because of this, proper wound preparation and care are critical to the management of wounds and the probability of a successful recovery. Typical wound care includes the removal of foreign matter, debris and necrotic tissue; the application of a topical or systemic anti-infection drug; and the isolation of the wound using some type of dressing. However, it is often the case that even after the application of these accepted wound care techniques, foreign materials that can cause infections can remain at the wound site or subsequently contaminate the wound. These foreign materials may result in infections of varying degrees of severity.

Therefore, there is a need for a system and a method for treating wounds or lesions that minimizes contact between undesired foreign material and the wound or lesion, and inhibits the establishment of infection. It is noted that the present disclosure will refer to wounds and lesions. However, it is intended that these terms will also cover infection sites and potential infections sites, infected tissue, infected region, areas exposed to infectious agents.

Currently, the methods of treating an established surface or subsurface infection associated with a wound or lesion involve the topical or systemic administration of anti-infective agents to the subject. Antibiotics are one such class of agents. Unfortunately, an ever-growing number of infective agents such as bacteria are becoming increasingly resistant to conventional antibiotics. It is well documented that the increased use of antibiotics has led to a dramatic increase in drug-resistant strains of bacteria. There are a number of disadvantages associated with traditional treatments, including the cost, the method of delivery, the availability and storage, and the possibility of adverse reactions in the subject because of allergies or sensitivities to the drugs.

The conventional treatment of surface or subsurface infections is also rendered less effective because the infective agent may interfere with the blood circulation within the infected region. Sometimes an infective agent constricts the capillaries or other small blood vessels in the infected region, reducing the blood perfusion of the tissue. Impairing the circulation results in the delivery of a lower level of anti-infective agent to the infected region, possibly aggravating the infection or slowing the effects of the treatment. Consequently, the total amount of drug that must be administered to the subject must be increased, adding to the expense of using such drugs.

Therefore, there is a need for a system and a method for treating wounds or lesions that does not require a topical or systemic administration of anti-infective agents to the subject.

In the 1980's, researchers discovered that the endothelium tissue of the human body produces nitric oxide (NO), and that NO is an endogenous vasodilator, namely, an agent that widens the internal diameter of blood vessels. NO, most commonly known as an environmental pollutant, is produced as a byproduct of combustion. At high concentrations, NO is toxic. At low concentrations, however, it has been discovered that inhaled NO can be used to treat various pulmonary diseases in humans. For example, NO has been investigated for the treatment of increased airway resistance in humans as a result of emphysema, chronic bronchitis, asthma, adult respiratory distress syndrome (ARDS), and chronic obstructive pulmonary disease (COPD).

The inventors of the instant invention have investigated NO for its use as a sterilizing agent in the prevention and treatment of infections. Tests performed in vitro have shown that NO will interfere with the growth of and/or kill many types of bacteria. PCT International Application No. PCT/CA99/01123, a published Jun. 2, 2000, discloses a method and an apparatus for the treatment of human respiratory infections by NO inhalation. NO has been found to have either an inhibitory and/or a cidal effect on pathogenic cells.

The present inventors have also found promising results for NO with respect to certain medical applications; however, there are certain risks inherent with gaseous NO delivery that make it necessary to use specialized delivery methods and systems. First, exposure to high concentrations of NO is toxic. The Occupational Health and Safety Administration (OSHA) has set the Immediate Danger to Life and Health (IDLH) limit as 100 parts per million (ppm) for 30 minutes, meaning that one could be exposed to NO at a concentration of 100 ppm for a maximum of 30 minutes before the effects of the exposure would pose a threat to health or life. Even lower levels of NO can be harmful if the time of exposure is relatively long. OSHA has also set exposure limits for NO in the workplace at 25 ppm time weighted averaged for eight (8) hours. Because of the dangers associated with exposure to potentially lethal doses of NO, any device or system for delivering NO must include features to prevent the leaking of NO into the surrounding environment in a manner that may raise a risk that the leaked NO might be inhaled or otherwise undesirably applied to subjects that can be harmed by such exposure to NO.

Therefore, there is a need for a system and a method for treating wounds or lesions with topical application of NO yet without the just-described risks of exposure of subjects or others to NO.

Another problem posed by NO is its rapid oxidation in the presence of oxygen to form NO₂, a gas that is highly toxic even at low levels. Unacceptably high levels of NO₂ can form if a delivery device contains a leak; a sufficiently large or continuous leak will also significantly reduce the amount of NO available for the desired therapeutic effect. Factors that can affect the rate of oxidation of NO to NO₂ include the concentration of NO, the concentration of O₂, and the time available for reaction., Because NO will react with the oxygen in the Air to convert to NO₂, there should be minimal contact between the NO gas and the outside environment.

Therefore, there is a need for a system and a method for the prevention of infection in wounds or lesions by the topical application of NO yet without the risks of leaking NO into the surrounding environment in which NO can react with oxygen in sufficient quantities to result in dangerous levels of NO

### OBJECTS OF THE INVENTION

It is a main object of the present invention to provide a system and a method for treating wounds or lesions, which prevents undesired foreign matter from contacting the wound or lesion.

It is another main object of the present invention to provide a system and a method for treating wounds or lesions, which may eliminate the requirement for a topical or systemic administration of anti-infective agents to the subject.

It is another object of the present invention to provide a system and a method for treating wounds or lesions with topical application of NO yet without the above-described risks of undesired exposure of subjects or others to NO.

It is another object of the present invention to provide a system and a method for treating wounds or lesions with a topical application of NO-containing gas to a wound or lesion of tissue with the primary aim of preventing the introduction of infectious agents in a wound.

It is another object of the present invention to provide a system and a method for treating wounds or lesions by use of the topical delivery of NO to inhibit the growth of and/or kill bacteria, thereby preventing an infection from invading the tissue.

### SUMMARY OF THE INVENTION

These, and other, objects are achieved by a system and a method for topically applying NO-containing gas to a wound or lesion of tissue in a manner that is controllable and safe from undesired leakage. Application of topical NO enhances blood flow and inhibits and/or impedes the growth of or kills bacteria, preventing tissue damage.

The basic system embodying the present invention includes a flushing envelope attached to a patient in covering relation to a wound or lesion of the patient to be treated topically with NO-containing gas and which is fluidically connected to a source of NO-containing gas and to a vent. NO is applied to the wound and flushes through and out of the flushing envelope in controlled amounts and concentrations a flow control valve, and a vacuum unit.

The flushing envelope is sealed to the patient around the wound or lesion being treated whereby NO does not leak to the surrounding environment and undesired foreign matter is prevented from contacting the wound or lesion.

The system can include a vacuum unit as well as a flow control valve and system controller. A source of dilutant gas can also be included and is fluidically combined with the NO gas by a gas blender unit.

The vacuum unit is optional, in that it is required only for certain configurations. The flushing envelope may be configured to operate with the system at positive, neutral or negative pressures with respect to the surrounding atmosphere. The vacuum unit can be configured to reduce or prevent the undesired escape of NO around the seal areas and can remove Air that may enter. However, depending upon the nature and integrity of the flushing envelope seal to the tissue surface, the system could be operated without a vacuum unit and at a significant positive pressure while still preventing NO escape other than through the intended vent line.

The flow controller unit or controller can also control the operation of the flow control valve and the vacuum unit and blender unit via signal transmission and receipt elements as are known to those skilled in the art.

The system can also include a nitric oxide gas absorber unit if NO concentrations warrant it. The gas absorber unit can be located at any suitable location, but the preferred location is fluidically downstream of the flushing envelope. One embodiment of the system has the gas absorber unit located upstream of the vacuum unit.

The present invention also includes a method of delivering an effective amount of nitric oxide to wound site for the prevention of infection. The method includes forming a substantially airtight seal with the tissue to be treated, transporting gas containing nitric oxide to the tissue to be treated, bathing the wound site with gaseous nitric oxide, and evacuating at least a portion of the nitric oxide gas from the wound or lesion. The method further includes placing a flushing envelope around the wound or lesion and controlling the amount of concentration of nitric oxide applied to the wound or lesion.

The system and method of the present invention provides the prevention of surface and subsurface infections in patients by topical application of NO. The system is leak-proof to the greatest degree possible and thus avoids a dangerous build-up of NO and NO₂ concentrations in the area adjacent to the system. In addition, this system seals the application area on the patient such that delivery of NO to the infected region of a patient will not allow the introduction of Air that would otherwise react with NO to produce NO₂ or of any other undesired foreign material to the wound or lesion. The topical application of NO to the infected region prevents the onset of infection, thereby prohibiting the formation of bacterial agents in the infected tissue. One form of the system of the present invention includes NO and NO₂ absorbers or scrubbers that will remove of chemically alter NO and NO₂ prior to discharge of the Air from the delivery system, with the concomitant advantages associated with such removal.

### BRIEF DESCRIPTION OF THE DRAWINGS

FIG. 1A is a schematic representation of a system for treating a wound or lesion with nitric oxide.

FIG. 1B illustrates a schematic representation of one possible configuration of an NO delivery system embodying the present invention.

FIG. 2 illustrates a flushing envelope with a top seal surrounding a patient's leg.

FIG. 3 illustrates a flushing envelope with a top seal surrounding a patient's foot.

FIG. 4 illustrates a flushing envelope with a top seal and a bottom seal surrounding a patient's knee.

### DETAILED DESCRIPTION OF THE PREFERRED EMBODIMENT OF THE INVENTION

Other objects, features and advantages of the invention will become apparent from a consideration of the following detailed description and the accompanying drawings.

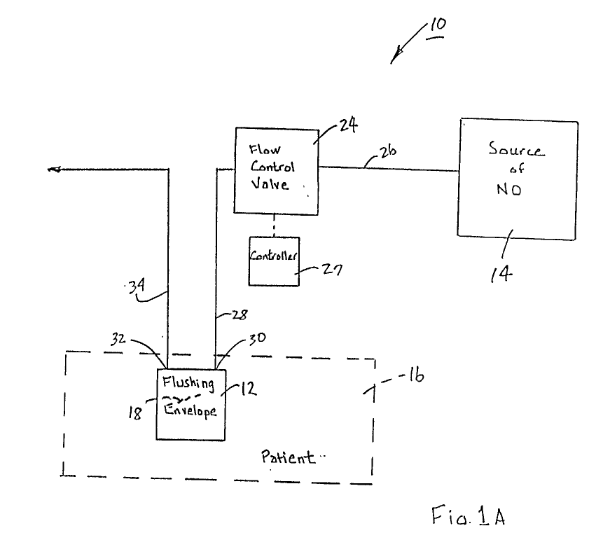

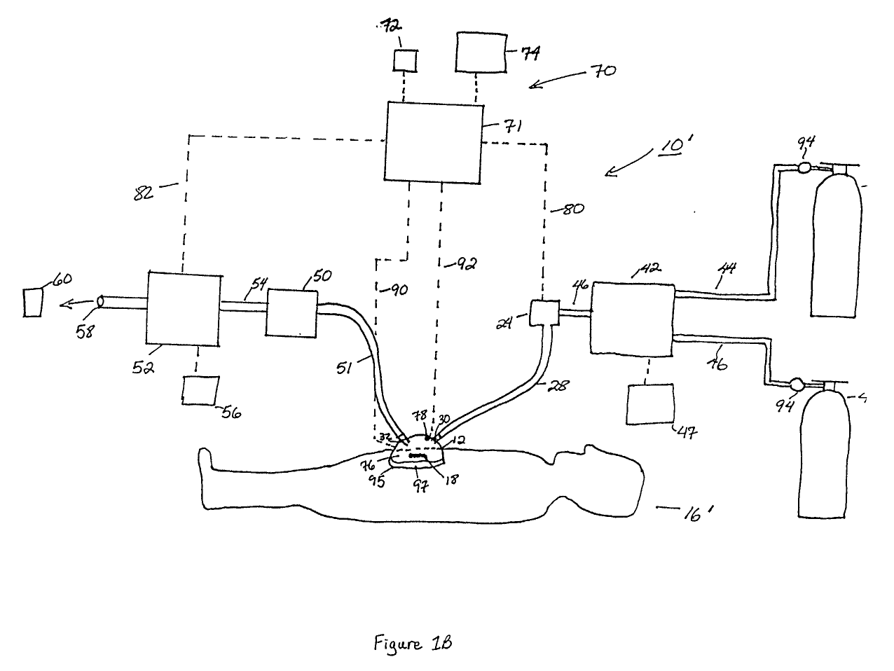

As discussed above, the inventor has discovered that wounds or lesions can be treated by applying nitric oxide to the site in a manner that controls the concentration of nitric oxide applied and prevents the nitric oxide from leaking into the environment surrounding the treatment area. In general, this is achieved by an NO delivery system 10 as shown in FIG. 1A. As shown in FIG. 1A, NO delivery system 10 includes a flushing envelope 12 fluidically connected to an NO gas source 14, and is mounted in place on a patient 16 over a wound or lesion 18 to apply NO to wound 18 on a patient.

More specifically, as shown in FIG. 1A, system 10 includes NO source 14 fluidically connected to flow control valve 24 by a conduit 26. Flow control valve 24 can include, for example, a proportional control valve that opens (or closes) in a progressively increasing manner (or decreasing manner if closing). As another example, flow control valve 24 can include a mass flow controller or be controlled by a suitable control unit 27. A conduit 28 fluidically connects flow control valve 24 to an inlet 30 of flushing envelope 12 for applying NO to wound 18. After flowing through flushing envelope 12, NO exits flushing envelope 12 via outlet 32 to which a conduit 34 is connected. Inlet 30 and outlet 32 can be one-way valves to control flow direction through envelope 12, and are spaced apart from each other to ensure a desired residence time of NO inside flushing envelope 12 adjacent to wound 18. Conduit 34 conducts NO away from envelope 12 to be properly disposed, as by venting either to the atmosphere in the appropriate circumstances or to a disposal container. It is noted that NO source 14 can include a pressurized cylinder or be a wall-located outlet as may be the situation in some hospitals. The spacing between inlet 30 and 32 can be taken into account when determining flow rates of NO through envelope 12 so the desired residence time is achieved.

An NO delivery system 10′ is shown in FIG. 1B and reference is made thereto. As shown in FIG. 1B, system 10′ includes a source 40 of dilutant gas such as N₂, O₂, Air or any other inert gas mixture blended with the NO in a gas blender 42. It is preferable to use a gas such as N₂ or an inert gas to dilute the NO concentration since these gases will not oxidize the NO into NO₂ as would O₂ or Air. While dilutant gas source 40 is shown as being a pressurized cylinder, other sources, including wall-located sources, can be used. Conduits 44 and 46 fluidically connect gas blender 42 with NO source 14 and dilutant gas source 40 respectively. A conduit 46 located fluidically downstream of gas blender 42 fluidically connects gas blender to flushing envelope 12 via flow control valve 24. Gas blender 42 uses membranes, controlled valves, or other suitable elements to blend NO with proper amounts of dilutant gas to achieve the desired concentration of NO to be applied to wound 18. A control element 47 can be connected to gas blender 42 where suitable.

As is also shown in FIG. 1B, system 10′ further includes an absorber unit 50 fluidically connected to flushing envelope 12 by a conduit 51. Absorber unit 50 preferably absorbs or strips NO from the gas stream flowing therethrough and that is exhausted from flushing envelope 12. It is also preferable for absorber unit 50 to also absorb or strip any NO from the gas stream that is exhausted from the flushing envelope 12. Since these gases are toxic at high levels, it is preferable that these components be removed from the NO delivery system prior to the gas being vented to the atmosphere. In addition, these gases can react with the internal components of the elements of the system and interface with the overall operation of the system. Absorber unit 50 can include various elements, such as membranes or the like, to absorb or remove NO and/or NO₂ from the gas stream. Those skilled in the art will understand what sort of gas absorbing elements will be used in absorber 50 based on the teaching of this disclosure.

After exiting absorber unit 50, the now clean gas flows from absorber unit 50 to a vacuum unit 52 via tubing 54. Vacuum unit 52 applies a negative pressure within the tubing to extract gases from flushing envelope 12. Vacuum unit 52 can be controlled by a controller 56 with respect to the level of vacuum or suction applied to tubing 54 and flushing envelope 12. In this regard, in conjunction with flow control valve 24, the amount of NO gas within flushing envelope 12 can be regulated. The gas then passes from vacuum unit 50 to a vent 58 that can be open to the atmosphere or can be fluidically connected to a suitable collection unit 60 if suitable.

It should be understood that absorbing unit 50 is an optional component of the delivery system. The gas laden with NO and NO₂ does not have to be removed from the gas stream if there is no concern with local levels of NO or NO₂. For example, the gas can be exhausted to the outside environment where high concentrations of NO and NO₂ will not develop (such as well-ventilated areas for example). Alternatively, a recirculation system (not shown) might be fluidically connected back to envelope 12 to recycle NO within flushing envelope 12.

Still referring to FIG. 1B, delivery system ′ includes a control unit 70 that is capable of controlling flow control valve 24 and vacuum unit 50. Control unit 70 includes a main control console 71 that includes suitable chips, microprocessors, setting controls and the like, and further includes an input device 72. Input device 72 is used by an operator to adjust various parameters of the delivery system such as NO concentration, residence time of NO, pressure within flushing envelope 12, and the like. An optional readout 74 can also be included in control unit 70 to display measured parameters and settings such as the set point of NO concentration, the concentration of NO within flushing envelope 12, the concentration of NO₂ within flushing envelope 12, the flow rate of gas into flushing envelope 12, the flow rate of gas out of the flushing envelope 12, the total time of delivery, and the like. Suitable pressure and flow sensors can also be located throughout the system, such as on various conduits and tubing to assist this control function. Temperature sensors can also be used where and when appropriate. Control unit 70 preferable receives the signals from sensors 76 and 78 located in flushing envelope 12 regarding gas concentrations of NO and NO₂ respectively. Signal lines 80 and 82 connect control unit 70 to flow control valve 24 and vacuum unit 52 respectively for delivery and receipt of control signals. NO sensor 76 is located in flushing envelope 12 and reports information regarding the concentration of NO in flushing envelope 12 to controller 70 via signal connection 90, typically a wired connection but possibly connected by other means, including over-the-air communication. NO₂ sensor 78 is located in the flushing envelope 12 and reports concentration of NO₂ in the flushing envelope to controller 70 via a signal connection 92, which can also be over-the-air if desired. Sensors 76 and 78 can be chemiluminescence-type, electromechanical cell-type, or spectrophotometric-type or other technologies suitable for the detection of NO and/or NO₂. Any suitable transmitter/receiver system can be used to couple controller 70 to the elements of the NO delivery system without departing from the scope of the present disclosure.

In another form of the invention, control unit 70 can be eliminated. In this regard, the flow rate of the gas into the flushing envelope 12 and the flow rate of gas out of flushing envelope 12 are pre-set or adjusted manually. For example, an operator can set a vacuum output that is substantially equal to the flow rate of the gas delivered to flushing envelope 12 via the flow control valve 24. In this manner, NO gas will be able to bathe the wound or lesion 18 without any build-up or leaking of NO or NO₂ gas from the delivery system.

It is noted that while N₂ is typically used to dilute the concentration of NO within a pressurized cylinder, any inert gas can be used without departing from the scope of the present disclosure. When the NO gas source is stored in a pressurized cylinder, it is preferable that the concentration of NO in the pressurized cylinder fall within the range provided by commercial nitric oxide manufacturers. Typically, nitric oxide mixtures for medical use are found at around the 1000 ppm range. High concentrations of NO are less desirable because accidental leakage of NO gas can be more hazardous, high partial pressures of NO tend to cause the spontaneous degradation of NO into NO₂ and the accurate delivery of the corresponding small volumes or flowrates is more difficult. Pressurized cylinders containing low concentrations of NO (i.e. less than 100 ppm NO) can also be used in accordance with the system as long as the concentration is sufficient to produce the desired cidal effect consistent with the methods disclosed herein. Concentrations of less than 200 ppm NO can also be used in the proper situations.

The NO gas from NO gas source 14 and the dilutant gas from dilutant gas source 40 preferably pass through pressure regulators 94 or other suitable flow and pressure control devices to reduce the pressure of gas that is admitted to the NO delivery system. Preferably, the NO-containing gas that is output from gas blender 42 has a concentration that is less than about 100 ppm or 200 ppm.

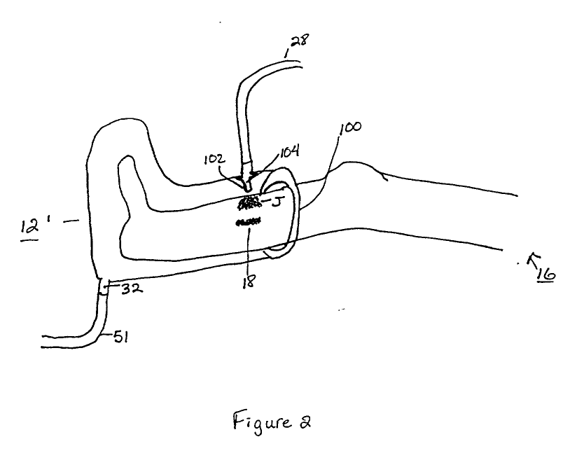

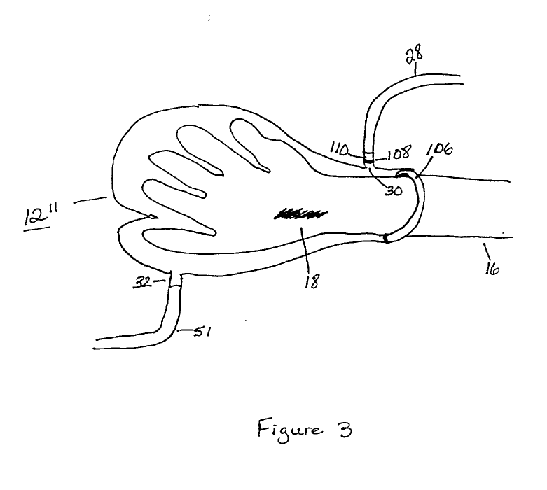

Still referring to FIG. 1B, the flushing envelope 12 is shown sealed against the tissue surface of a patient 16′. Wound or lesion 18, which can be an abscess, lesion or wound or the like, is enclosed by the flushing envelope 12. Flushing envelope 12 preferably includes a seal portion 95 that forms a substantially airtight seal with the tissue of the subject. ‘Substantially air-tight’ is meant to indicate that the NO-containing gas does not leak out of the flushing envelope 12 in significant amounts i.e., no more than about 5% of the NO-containing gas delivered to the flushing envelope 12 in significant amounts. Seal portion 95 may comprise an inflatable seal 97, such as that shown in FIGS. 2 and 3, or alternatively the seal portion 95 may comprise a flexible skirt or the like that conforms to the tissue surface of the subject. Seal portion 95 can also include an adhesive portion that adheres to the tissue surface of the subject. In other envisioned embodiments, the sealing portion 95 may merely comprise the interface of the flushing envelope 12 with the surface of the tissue.

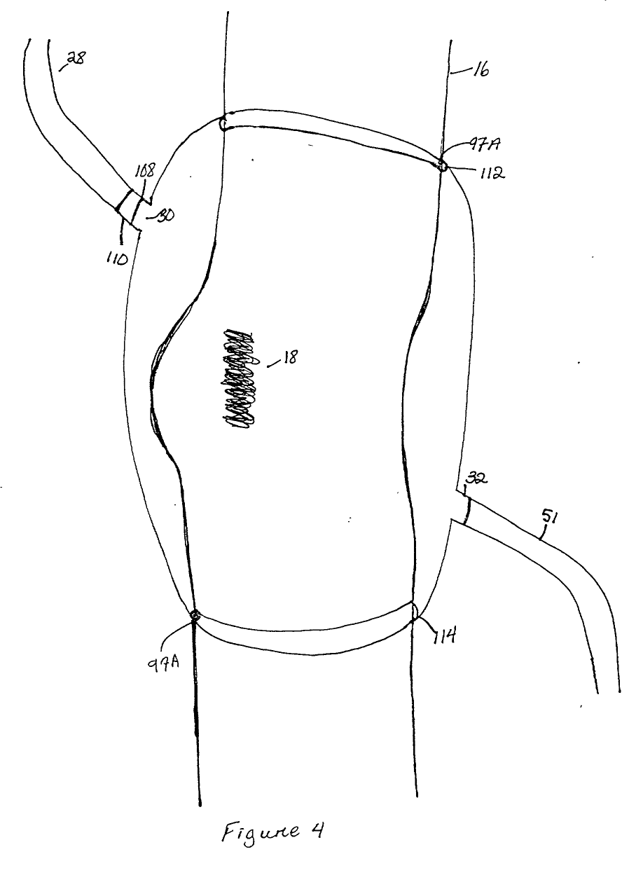

The flushing envelope 12 can be made of a virtually limitless number of shapes and materials depending on its intended use. The flushing envelope 12 might be formed as a rigid structure, such as that shown in FIG. 1B, that is placed over the wound or lesion. Alternatively, the flushing envelope 12 can be formed of a flexible, baglike material that is inflatable over the wound or lesion. FIG. 2 shows such a structure 12′ in the shape of a boot placed over a patient's leg. FIG. 3 shows an inflatable flushing envelope 12 that is formed in the shape of a mitten or covering that is worn over, for example, a patient's hand. FIG. 4 shows another flushing envelope 12′″ in the form of a sleeve or cuff that is fitted over the knee of a patient, sealed by adhesive 97A at the top and bottom, such that the patient does not stand on the material of the flushing envelope. The flushing envelope can also include translucent or transparent material so the wound can be viewed through the flushing envelope. In this way, the healing process can be monitored.

As just mentioned, flushing envelope 12′ shown in FIG. 2 is in the shape of a boot used to treat a wound or lesion located on the leg of a patient. The flushing envelope 12′ includes an inflatable seal that surrounds the leg region to make a substantially airtight seal with the tissue. This embodiment includes an inlet flow control element, such as a nozzle 102 that is affixed near the inlet 104 of the flushing envelope 12′. The nozzle 102 directs a jet of gaseous NO onto the wound or lesion. The jet of gaseous NO aids in penetrating the wound or lesion to prevent the establishment of pathogens. As will occur to those skilled in the art based on the teaching of the present disclosure, other inlet flow control elements, such as orifices, valves, or the like, can be used in place of nozzle 102 without departing from the scope of the present disclosure.

As also discussed above, the flushing envelope can be in the shape of a mitten. As shown in FIG. 3, mitten-shaped flushing envelope 12″ is inflatable and contains inflatable seal 106 that forms a substantially airtight seal around the tissue of a patient. FIG. 3 also shows an optional one way valve 108 located in the inlet 110 of flushing envelope 12″. As seen, the inlets and outlets are located spaced apart from one another, and preferably on opposing sides of the treated area such that freshly-delivered NO gas is not prematurely withdrawn from the flushing envelope.

FIG. 4 shows a flushing envelope 12′″ which is in the shape of a tube. Flushing envelope 12′″ is also inflatable and contains two inflatable seals at the top 112 and bottom 114 that form a substantially air-tight seal around the tissue of the patient.

For the prevention of infection, the flushing envelope 12′″ is placed over the wound or lesion. An airtight seal is then formed between the tissue of the patient and the flushing envelope. If the flushing envelope 12′″ has an inflatable construction, the flushing envelope must be inflated with gas. Preferably, the flushing envelope 12′″ is initially inflated only with the dilutent gas to prevent the leaking of NO and NO₂ from the system. Once an adequate airtight seal has been established, the operator of the device initiates the flow of NO from the NO gas source to the flushing envelope. As described above, this may be accomplished manually or via the controller 70.

Referring back to FIG. 1B, the preferred method of treating a wound or lesion 18 will now be described. Flushing envelope 12 is placed over the wound or lesion and an airtight seal is formed between the tissue of the patient and the flushing envelope. If the flushing envelope has an inflatable construction, the flushing envelope must be inflated with gas. Preferably, the flushing envelope is initially inflated only with dilutant gas to prevent the leaking of NO and NO₂ from the system. Once an adequate airtight seal has been established, the operator of the system initiates the flow of NO from the NO gas source to the flushing envelope. As described above, this may be accomplished manually or via control unit 70.

Once the flushing envelope has started to fill with NO gas, the vacuum unit is turned on and adjusted to the appropriate output level. For an inflatable flushing envelope, the output level (i.e., flow rate) of the vacuum unit should be less than or equal to the flow rate of NO gas entering the flushing envelope to avoid deflating the flushing envelope. In embodiments of the system where the flushing envelope is rigid, the vacuum unit can be set to create a partial vacuum within the flushing envelope. In this regard, the partial vacuum helps to form the airtight seal between the tissue and the flushing envelope. Of course, the vacuum unit can also be set to withdraw gas at a substantially equal rate as the gas is delivered to the flushing envelope if suitable. An effective amount of NO is delivered to the flushing envelope to prevent the growth of pathogens in the wounded area. Pathogens include bacteria, viruses, and fungi.

It is understood that while certain forms of the present invention have been illustrate and described herein, it is not to be limited to the specific forms or arrangements of parts described and shown

1. A method of treating wounds or lesions in patients comprising: the topical application of NO to a wound or lesion to prevent infections and speed healing.

2. The system for the topical delivery of nitric oxide gas to a potential infection site that may subsequently be exposed to infectious agents comprising:
a source of nitric oxide gas;
a flushing envelope in fluid communication with said source of nitric oxide, said flushing envelope including a seal which forms a seal with a patient's tissue when said flushing envelope is in place on the patient; and
a flow control valve positioned fluidically downstream of said source of nitric oxide and fluidically upstream of said flushing envelope and fluidically connected to said flushing envelope and controlling the amount of nitric oxide gas delivered to said flushing envelope.

3. The system defined in claim 2 further comprising a vacuum unit in fluid communication with said flushing envelope and positioned fluidically downstream of said flushing envelope and withdrawing gas from said flushing envelope.

4. The system defined in claim 2 further comprising a gas blender located fluidically upstream of said flow control valve and fluidically connected to said source of nitric oxide gas and to a source of dilutant gas and including means for mixing nitric oxide gas with dilutant gas.

5. The system defined in claim 3 further comprising an absorber unit located fluidically upstream of said vacuum unit and including means for removing nitric oxide from a gas stream flowing through said absorber unit.

6. The system defined in claim 5 wherein the absorber unit also includes means for removing nitrogen dioxide from the gas stream flowing through said absorber unit.

7. The system defined in claim 2 wherein said seal of said flushing envelope includes an inflatable seal.

8. The system defined in claim 2 wherein said flushing envelope includes an inflatable material.

9. The system defined in claim 2 further comprising a gas nozzle located inside said flushing envelope and directing gas at a wound or lesion.

10. The system defined in claim 2 wherein said source of nitric oxide includes a pressurized cylinder containing nitric oxide.

11. The system defined in claim 2 further comprising a controller for controlling the operation of the flow control valve.

12. The system defined in claim 2 further comprising a nitric oxide sensor located within said flushing envelope.

13. The system defined in claim 2 further comprising a nitrogen dioxide sensor located within the flushing envelope.

14. The system defined in claim 2 wherein said flushing envelope comprises an inflatable bag.

15. The system defined in claim 2, wherein said flushing envelope contains less than 800 ppm nitric oxide when positioned on a wound or lesion during the treatment of said wound or lesion.

16. The system defined in claim 15 wherein said flushing envelope contains less than 100 ppm of nitric oxide when positioned on a wound or lesion during treatment of said wound or lesion.

17. The system defined in claim 1 further including a step of forming an air-tight seal around a wound or lesion being treated.

18. A system for the topical delivery of nitric oxide gas to a potential infection site that may subsequently be exposed to infectious agents, the device comprising:
source of nitric oxide gas;
source of dilutant gas;
gas blender in fluid communication with said source of nitric oxide gas and said source of dilutant gas, said gas blender including means for blending nitric oxide gas and dilutant gas to form an output mixture containing a nitric oxide gas mixture;
a flow control valve in fluid communication with an output of said gas blender;
a flushing envelope in fluid communication with said flow control valve, said flushing envelope including an input and an output, the input being in fluid communication with the output of said flow control valve, said flushing envelope having seal portions that contact tissue adjacent to an area being treated and forming an air-tight seal with that tissue when said flushing envelope is in place on a patient;
a nitric oxide gas absorber unit disposed fluidically downstream of said flushing envelope and fluidically connected to said flushing envelope; and
a control unit connected to said flow control valve.

19. The system defined in claim 18 further including a vacuum unit in fluid communication with the output of said flushing envelope.

20. The system defined in claim 18 wherein said absorber unit includes means for removing nitrogen dioxide from a gas stream passing through said absorber unit.

21. The system defined in claim 18 further including an inflatable seal on said flushing forming an airtight seal with the tissue when said flushing envelope is in place.

22. The system defined in claim 18 wherein said flushing envelope includes inflatable material.

23. The system defined in claim 18 further comprising a gas nozzle located inside said flushing envelope and directing gas at a wound or lesion when said flushing envelope is in place.

24. The system defined in claim 18 wherein said source of nitric oxide includes a pressurized cylinder containing nitric oxide.

25. The system defined in claim 18 further including a nitric oxide sensor located within said flushing envelope.

26. The system defined in claim 18 further comprising a nitrogen dioxide sensor located within said flushing envelope.

27. The system defined in claim 18 wherein said flushing envelope includes an inflatable bag.

28. The system defined in claim 18 wherein said flushing envelope contains less than 800 ppm nitric oxide when positioned on a wound or lesion during treatment of said wound or lesion.

29. The system defined in claim 18 wherein said flushing envelope contains less than 100 ppm nitric oxide when positioned on a wound or lesion during the treatment of said wound or lesion.

30. The system defined in claim 18, wherein the flushing envelope includes a seal mechanism having separated segments that isolate a wound or lesion.

31. A method of delivering an effective amount of nitric oxide to a potential infection site that may subsequently be exposed to infectious agents, to reduce pathogen levels comprising the steps of:
placing a flushing envelope around a wound or lesion;
forming a substantially air-tight seal with the tissue using the flushing envelope; and
transporting a gas containing nitric oxide to the flushing envelope and bathing the wound or lesion with nitric oxide.

32. The method defined in claim 31 further including a step of evacuating at least a portion of the nitric oxide gas from the flushing envelope.

33. The method defined in claim 31 further including the steps of venting gas containing nitric oxide from the flushing envelope and removing at least a portion of the nitric oxide contained within the gas vented from the flushing envelope.

34. The method defined in claim 31 wherein the step of transporting gas further includes a step of controlling the flow rate of gas transported into the flushing envelope.

35. The method defined in claim 31 wherein the step of transporting gas further includes a step of directing the gas onto a the wound or lesion.

36. The method defined in claim 31 further including a step of forming a seal mechanism separated segments that isolate a wound or lesion.

37. A system for the topical delivery of nitric oxide gas to a potential infection site that may subsequently be exposed to infectious agents comprising:
a source of nitric oxide gas;
a flow control valve in fluid communication with the source of nitric oxide gas;
a flushing envelope in fluid communication with an output of said flow control valve, said flushing envelope having portions that contact tissue around a wound or lesion that may subsequently be exposed to infectious agents when said flushing envelope is in place; and
a control unit connected to said control valve.

38. The system defined in claim 37 further comprising a vacuum unit fluidically connected to said flushing envelope when said flushing envelope is in place.

39. The system defined in claim 37 further comprising a source of dilutant gas and a gas blender located upstream of said flow control valve and fluidically connected to said source of nitric oxide gas and said to be a source of dilutant gas and having means for mixing nitric oxide gas and dilutant gas together.

40. The system defined in claim 38, further comprising an absorber unit disposed fluidically upstream of said vacuum unit and having means for removing nitric oxide from a gas stream passing through said absorber unit.

41. The system defined in claim 40 wherein said absorber unit further includes means for removing nitrogen dioxide from the gas stream passing through said absorber unit.

42. The system defined in claim 37, wherein said flushing envelope further includes an inflatable seal.

43. The system defined in claim 37, wherein said flushing envelope further includes an inflatable material.

44. The system defined in claim 37 further comprising a gas nozzle located inside said flushing envelope.

45. The system defined in claim 37 wherein said source of nitric oxide includes a pressurized cylinder containing nitric oxide.

46. The system defined in claim 37 further comprising a nitric oxide sensor located within said flushing envelope.

47. The system defined in claim 37 further comprising a nitrogen dioxide sensor located within said flushing envelope.

48. The system defined in claim 37 wherein said flushing envelope includes an inflatable bag.

49. The system defined in claim 37 wherein said flushing envelope contains an effective amount of nitric oxide when positioned on a wound or lesion.

50. The system defined in claim 37 wherein the total seal mechanism includes one or more separated segments that isolate a wound or lesion.

51. A method of preventing infectious pathogens to infect a wound or lesion comprising:
placing a flushing envelope over a wound or lesion;
forming an air-tight seal between tissue of a patient and the flushing envelope;
inflating the flushing envelope with gas, including steps of initially inflating the flushing envelope only with dilutant gas and establishing an adequate air-tight seal between the flushing envelope and a patient;
initiating flow of NO to the flushing envelope;
once the flushing envelope has started to fill with NO gas, turning on a vacuum unit and adjusting the vacuum unit to establish a desired flow rate of gas through the flushing envelope; and
delivering an effective amount of NO to the flushing envelope to prevent pathogens from establishing themselves in the wound or lesion.

52. The method defined in claim 51 further including a step of establishing a vacuum to adjust the flow through the flushing envelope so the flow rate is less than the flow rate of NO gas entering the flushing envelope.

53. The method defined in claim 51 including a step of setting the vacuum unit to withdraw gas at a substantially equal rate as the gas is delivered to the flushing envelope to establish a steady flow rate through the flushing envelope.

54. The method defined in claim 1 further including a step of preventing NO from venting to the atmosphere adjacent to the patient.

55. The method defined in claim 53 further including a step of preventing evironmental air from contacting the NO being applied to the wound or lesion.
